(19) United States
(12) Patent Application Publication
(10) Pub. No.: US 2014/0155608 A1
(43) Pub. Date: Jun. 5, 2014

(54) METHOD FOR THE MANUFACTURING OF NALTREXONE

(71) Applicant: H.LUNDBECK A/S, Valby (DK)

(72) Inventors: Carla De Faveri, Farra di Soligo (TV) (IT); Mariano Stivanello, Schio (VI) (IT); Florian Anton Martin Huber, Dolo (VE) (IT)

(73) Assignee: H. LUNDBECK A/S

(21) Appl. No.: 13/875,351

(22) Filed: May 2, 2013

Related U.S. Application Data
(60) Provisional application No. 61/642,490, filed on May 4, 2012.

(30) Foreign Application Priority Data
May 3, 2012 (DK) PA 2012 00303

(51) Int. Cl. C07D 489/08

(52) U.S. Cl. CPC C07D 489/08
USPC 546/45; 546/44

(57) ABSTRACT

The present invention relates to a new process for producing naltrexone [17-(cyclopropylmethyl)-4,5α-epoxy-3,14-dihydroxy-morphinan-6-one] from noroxymorphone [4,5-α-epoxy-3,14-dihydroxy-morphinan-6-one] by alkylation with a cyclopropylmethyl halide.

### FIELD OF THE INVENTION

The present invention relates to a new process for producing naltrexone [17-(cyclopropylmethyl)-4,5α-epoxy-3,14-dihydroxy-morphinan-6-one] from noroxymorphone [4,5-α-epoxy-3,14-dihydroxy-morphinan-6-one] by alkylation with a cyclopropylmethyl halide.

### BACKGROUND OF THE INVENTION

Nalmefene is a known opioid system modulator, with a distinct μ, δ, and κ receptor profile, which can inhibit pharmacological effects of both administered opioid agonists and endogenous agonists derived from the opioid system. The clinical usefulness of nalmefene comes from its ability to promptly (and selectively) reverse the effects of these opioid agonists.

Nalmefene has primarily been developed as the hydrochloride salt for use in the management of alcohol dependency, where it has shown good effect at doses of 10 to 40 taken when the patient experiences a craving for alcohol (Karhuvaara et al., Alcohol. Clin. Exp. Res., (2007), Vol. 31 (7): 1179-1187; Trial watch: Nalmefene reduces alcohol use in phase III trial, Nature reviews Drug discovery (2011) Vol. 10 (8): 566). Additionally, nalmefene has also been investigated for the treatment of other addictions such as pathological gambling and addiction to shopping.

Nalmefene is an opiate derivative structurally related to the opiate antagonist naltrexone. Advantages of nalmefene compared to naltrexone include longer half-life, higher oral bioavailability and the absence of dose-dependent liver toxicity.

Nalmefene can be produced from naltrexone by the Wittig reaction. Methods for preparation of nalmefene from naltrexone by the Wittig reaction has been described by Hahn at al., (J. Med. Chem. (1975) Vol. 18: 259-282, Mallinckrodt (U.S. Pat. No. 4,751,307), Meltzner et al., (U.S. Pat. No. 4,535,157) and by H. Lundbeck (WO 2010/136039). By using the above-mentioned methods, the free base of nalmefene is obtained, which subsequently can be converted into the hydrochloride salt, by use of conventional methods.

Naltrexone can be produced from noroxymorphone by various direct and indirect alkylation methods. One method is by direct alkylation of noroxymorphone with cyclopropylmethylbromide. This process has been disclosed in general terms by Rice in WO 91/0576 Sanofi-Avensis (WO 2008/034973) describes a process for obtaining naltrexone in 88.6% yield by reacting noroxymorphone hydrochloride with cyclopropylmethylbromide in dimethylacetamide in the presence of sodium hydrogen carbonate. Cilag (WO 2008/138605) describes N-alkylation of noroxymorphone with cyclopropylmethylbromide in N-methyl-pyrrolidone in the presence of sodium hydrogen carbonate. Mallinckrodt (WO 2010/039209) describes N-alkylation of noroxymorphone with cyclopropylmethylbromide in the presence of a protic solvent. Specific examples in WO 2010/039209 describe the addition of water, iso-propanol or ethanol as the protic solvent.

There is a need within the field to improve the method of producing highly pure naltrexone and/or to find alternative processes for producing naltrexone. In particular, there is a need for a method that is readily applicable on industrial scale.

### SUMMARY OF THE INVENTION

The present invention relates to a new process for producing naltrexone [17-(cyclopropylmethyl)-4,5α-epoxy-3,14-dihydroxy-morphinan-6-one] from noroxymorphone [4,5-α-epoxy-3,14-dihydroxy-morphinan-6-one] by alkylation of noroxymorphone with a cyclopropylmethyl halide in N-methyl-2-pyrrolidone and certain amounts of water as depicted in scheme 1 below. The total amount of water compared to noroxymorphone is from 0.4 to 4.0 equivalents.

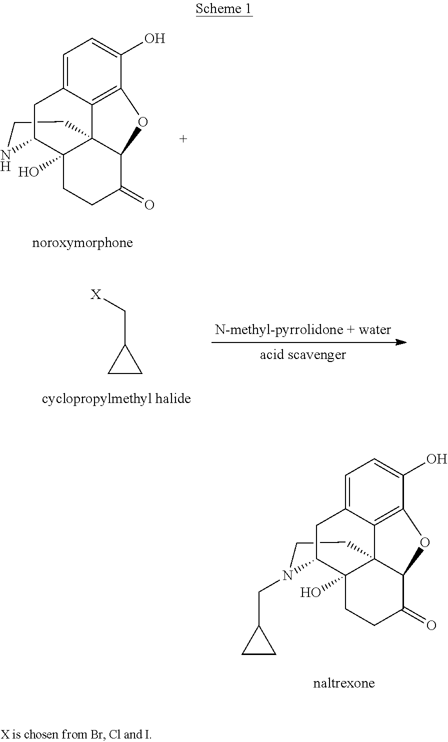

In one embodiment, naltrexone obtained from the process of the invention is further processed e.g. by the Wittig reaction to nalmefene.

In one embodiment the invention relates to a process for the manufacturing of nalmefene comprising the steps, i) manufacturing of naltrexone by a process of the invention, ii) further processing of naltrexone obtained from i) to nalmefene optionally by the Wittig reaction.

### DEFINITIONS

Throughout the description, the terms “naltrexone” and “nalmefene” are intended to include any forms of the compounds, such as the free base and pharmaceutically acceptable salts. The free base and pharmaceutically acceptable salts include anhydrous forms and solvated forms such as hydrates. The anhydrous forms and the solvates include amorphous and crystalline forms. In a particular embodiment naltrexone is in the form of the free base. In a particular embodiment nalmefene is in the form of the hydrochloride.

In the present context, examples of “cyclopropylmethyl halides” include cyclopropylmethyl bromide, cyclopropylmethyl chloride and cyclopropylmethyl iodide. In a particular embodiment, the term “cyclopropylmethyl halide” refers to cyclopropylmethyl bromide.

In the present context, an “acid scavenger” refers to a compound selected from organic and inorganic bases, and combinations hereof. Examples include borate salts, phosphate salts, bicarbonate salts (such as KHCO₃, NaHCO₃, LiHCO₃ and the like), carbonate salts (such as K₂CO₃, Na₂CO₃, Li₂CO₃ and the like), organic bases (such as pyridine, triethylamine tributylamine, N,N-diisopropylethylamine, N-methylmorpholine, N,N-dimethylaminopyridine), and mixtures of any of the above. In a particular embodiment the term “acid scavenger” refers to KHCO₃.

In the present context, the “total amount of water” in the process indicates the sum of water added to the process and water bound in the noroxymorphone starting material. In the examples of the current invention the amount of water in the noroxymorphone starling material has been determined by Karl Fisher titration (KF). When an amount of water is stated in equivalents, the equivalent indicates the molar amount of water relative to the molar amount of noroxymorphone.

In the present context, the term “chemically pure” has its normal meaning within the art. Accordingly, an obtained compound which is at least 98% chemically pure comprises at most 2% chemical impurities. The chemical purity may be determined e.g. by HPLC. In the present context chemical purity is determined by % HPLC area.

### DETAILED DESCRIPTION OF THE INVENTION

The inventors have found an improved process for producing naltrexone [17-(cyclopropylmethyl)-4,5α-epoxy-3,14-dihydroxy-morphinan-6-one] from noroxymorphone [4,5-α-epoxy-3,14-dihydroxy-morphinan-6-one] by alkylation with a cyclopropylmethyl halide in the presence of N-methyl-2-pyrrolidone and water. It has been experienced that the noroxymorphone starting material often contains up to a few equivalents of water. The amount of water bound to noroxymorphone is variable and can depend e.g. on the synthesis and work-up process of noroxymorphone. The inventors have found that when running the alkylation in N-methyl-2-pyrrolidone and certain equivalents of water, naltrexone is obtained as a chemically pure compound in a high yield. Since the total amount of water has an influence on the conversion of noroxymorphone to naltrexone and purity of the obtained naltrexone it is essential to balance the amount of added water with the amount of water in the noroxymorphone starting material. The best results are obtained when the total amount of water is from 0.4 to 4.0 equivalents, preferably from 0.7 to 3.5 equivalents, more preferably from 1.0 to 3.0 equivalents. Table 2 illustrates the relationship between obtained purity and total amount of water.

In brief, noroxymorphone is mixed with cyclopropylmethyl halide in N-methyl-2-pyrrolidone and water. In a preferred embodiment, the reaction is conducted in presence of an acid scavenger. The mixture is heated to a temperature in the range of 30 to 100° C., preferably in the range of 50-70° C. such as in the range of 55-60° C. Reaction time is adjusted in order to have a reasonably high conversion. Optionally, further cyclopropyl methyl halide is added to the mixture.

The formed naltrexone is isolated by a method comprising the following steps:

a) concentrating the reaction mixture under vacuum;

b) mixing the reaction mixture with an acid

c) mixing the resulting mixture with water

d) mixing the resulting mixture with a base

e) isolating the resulting solid.

f) drying the solid.

The process of the present invention consistently gives pure naltrexone. The main impurity coming from alkylation of the hydroxyl group in the phenolic moiety is controlled with the process of the invention. The level of the impurity 3-cyclopropylmethylnaltrexone in the isolated naltrexone is generally below about 0.5% by area) as measured by HPLC. The process of the invention also allows efficient removal of potentially unreacted noroxymorphone in the isolated naltrexone.

Naltrexone prepared according to the method described in this invention can thus be directly used in the preparation of nalmefene e.g. by Wittig reaction. It is also envisaged in the present invention that such obtained nalmefene can be transformed into a suitable pharmaceutically acceptable salt form such as the hydrochloride salt. In a particular embodiment nalmefene hydrochloride is obtained as dihydrate form. Nalmefene can be obtained from naltrexone by the Wittig reaction and transformed into the hydrochloride salt e.g. as disclosed in WO 2010/136039 and further transformed to nalmefene hydrochloride dihydrate as disclosed in WO 2010/063292.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and the and similar referents in the context of describing the invention are to be construed to cover both the singular and the plural, unless otherwise indicated herein or clearly contradicted by context. For example, the phrase “the compound” is to be understood as referring to various “compounds” of the invention or particular described aspect, unless otherwise indicated.

The description herein of any aspect or aspect of the invention using terms such as “comprising”, “having,” “including” or “containing” with reference to an element or elements is intended to provide support for a similar aspect or aspect of the invention that “consists of”, “consists essentially of” or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

It should be understood that the various aspects, embodiments, implementations and features of the invention mentioned herein may be claimed separately, or in any combination.

### EMBODIMENTS ACCORDING TO THE INVENTION

In the following, embodiments of the invention are disclosed. The first embodiment is denoted E1, the second embodiment is denoted E2 and so forth.
E1. A process for the manufacturing of naltrexone, comprising reacting noroxymorphone with cyclopropylmethyl halide in the presence of N-methyl-2-pyrrolidone and water, wherein the total amount of water compared to noroxymorphone is from 0.4 to 4.0 equivalents.
E2. The process according to embodiment 1, wherein the total amount of water compared to noroxymorphone is from 0.7 to 3.5 equivalents.
E3. The process according to embodiment 2, wherein the total amount of water compared to noroxymorphone is from 1.0 to 3.0 equivalents such as from 1.0 to 2.5 equivalents.
E4. The process according to any of embodiments 1-3, wherein the reaction takes place in the presence of an acid scavenger.
E5. The process according to embodiment 4, wherein the acid scavenger is an inorganic or organic base or a mixture thereof.
E6. The process according to any of embodiments 4-5, wherein the acid scavenger is a bicarbonate salt such as potassium bicarbonate.
E1. The process according to any of embodiments 16, wherein the cyclopropylmethyl halide is cyclopropylmethyl bromide.
E8. The process according to any of embodiments 1-7, wherein N-methyl-2-pyrrolidone is used in a weight by weight ratio of 0.5:1 to 10:1 with respect to noroxymorphone.
E9. The process according to embodiment 8, wherein N-methyl-2-pyrrolidone is used in a weight by weight ratio of 1:1 to 5:1 with respect to noroxymorphone.
E10. The process according to embodiment 9, wherein N-methyl-2-pyrrolidone is used in a weight by weight ratio of about 3:1 with respect to noroxymorphone.
E11. The process according to any of embodiments 3-10, wherein the molar relationship between noroxymorphone and acid scavenger is from 1:0.5 to 1:2.
E12. The process according to embodiment 11, wherein the molar relationship between noroxymorphone and acid scavenger is from 1:1 to 1:2.
E13. The process according to embodiment 12, wherein the molar relationship between noroxymorphone and acid scavenger is from 1:1 to 1:1.5.
E14. The process according to any of embodiments 1-13, wherein the molar relationship between noroxymorphone and cyclopropylmethyl halide is from 1:1 to 1:2.
E15. The process according to embodiment 14, wherein the molar relationship between noroxymorphone and cyclopropylmethyl halide is from 1:1 to 1:1.5.
E16. The process according to any of embodiments 1-15, wherein the reaction temperature is in the range of 30-100° C.
E17. The process according to embodiment 16, wherein the reaction temperature is in the range of 50-70° C., such as in the range of 50-55° C. or 55-60° C. or 60-65° C. or 65-70° C.
E18. The process according to any of embodiments 16-17, wherein the reaction temperature is in the range of 55-60° C.
E19. The process according to any of embodiments 1-18, wherein the reaction is running for at least 8 hours; such as in the range of 8-48 hours, such as 8-12 hours, 12-16 hours, 16-20 hours, 20-24 hours, 24-28 hours, 28-32 hours, 32-36 hours, 36-40 hours, 40-44 hours or 44-48 hours.
E20. The process according to embodiment 19, wherein the reaction is running for a range of 12-40 hours.
E21. The process according to embodiment 20, wherein the reaction is running for a range of 16-30 hours.
E22. The process according to any of embodiments 1-21, wherein the formed naltrexone is isolated by a method comprising the following steps
   a) concentrating the reaction mixture under vacuum;
   b) mixing the reaction mixture with an acid
   c) mixing the resulting mixture with water
   d) mixing the resulting mixture with a base
   e) isolating the resulting solid.
   f) drying the solid.
E23. The process according to embodiment 22 wherein the acid in step b) is hydrochloric acid.
E24. The process according to any of embodiments 22-23, wherein the base in step d) is ammonium hydroxide.
E25. The process according to any of embodiments 1-24, wherein the formation of 3-cyclopropylmethyl-naltrexone is less than about 0.5% (by area).
E26. The process according to any of embodiments 1-25, wherein noroxymorphone is used as starting material in form of its free base or its hydrochloride salt.
E27. The process according to any of embodiments 1-26, wherein naltrexone is obtained as the free base.
E28. The process according to embodiment 27, wherein naltrexone free base is obtained as a hydrate.
E29. The process according to embodiment 28, wherein the naltrexone free base hydrate is a monohydrate.
E30. The process according to embodiment 29, wherein the naltrexone free base monohydrate is obtained in crystalline form.
E31. The process according to any of embodiments 1-30, wherein the naltrexone obtained from the process is further processed to give nalmefene.
E32. The process according to embodiment 31, wherein naltrexone obtained from the process is further processed by the Wittig reaction to give nalmefene.
E33. A process for the manufacturing of nalmefene comprising the steps
   i) manufacturing of naltrexone by a process according to any of embodiments 1-30
   ii) further processing of naltrexone obtained from i) to nalmefene, optionally by the Wittig reaction.
E34. The process according to embodiment 33 comprising the following subsequent step iii) precipitating nalmefene as a pharmaceutically acceptable salt, and optionally purifying the obtained nalmefene salt.
E35. The process according to any of embodiments 31-34, wherein nalmefene is obtained as a hydrochloride.
E36. The process according to embodiment 35, wherein nalmefene hydrochloride is obtained as a dihydrate.
E37. The process according to embodiment 36, wherein nalmefene hydrochloride dihydrate is obtained in crystalline form.
E38. Naltrexone directly obtained from the process according to any of embodiments 1-30.
E39. Nalmefene directly obtained from the process according to any of embodiments 31-37.
E40. A pharmaceutical composition comprising nalmefene obtained from the process according to any of embodiments 31-37.

### EXAMPLES

The invention will be illustrated by the following non-limiting examples

### HPLC Chromatographic Conditions.

| | |
|---|---|
| Column: | Zorbax Eclipse XDB C18, 150 × 4.6 mm, 5 μm |
| | or equivalent |
| Mobile Phase A: | Acetonitrile/Buffer 10:90 |
| Mobile Phase B: | Acetonitrile/Buffer 45:55 |
| Buffer: | 1.1 g of Sodium Octanesulfonate dissolved in 1 L |
| | of water, pH adjusted to 2.3 with H₃PO₄. |
| Column Temperature: | 35° C. |
| Detector: | UV at 230 nm |
| Flow: | 1.2 mL/min |
| Injection volume: | 10 μl |
| Time of Analysis: | 55 minutes |

TABLE 1
HPLC gradient

| Time | Mobile Phase A | Mobile Phase B |
|---|---|---|
| 0 | 100 | 0 |
| 45 | 0 | 100 |
| 47 | 100 | 0 |
| 55 | 100 | 0 |

### Example 1

Noroxymorphone (37.7 g, KF 2.4%, assay 90%), N-methyl-2-pyrrolidone (150 mL), potassium bicarbonate (15.5 g) and cyclopropylmethyl bromide (18.2 g) were charged in a jacketed reactor. The suspension was heated up to 55° C. for 20.5 hours. The composition of the reaction mixture was checked by HPLC (% by area) tin reacted noroxymorphone 4.5%, naltrexone 94.0%, 3-cyclopropylmethylnaltrexone 0.1%.

### Example 2

Noroxymorphone (5.0 g, KF 3.34%) and potassium bicarbonate (1.7 g) were suspended in mL of N-methyl-2-pyrrolidone. The mixture was heated up to 70° C. and cyclopropylmethyl bromide (2.73 g) was charged. The mixture was maintained at 70° C. for 19.5 hours. The HPLC analysis showed that the reaction was not complete. Further cyclopropylmethyl bromide (0.68 g) was added and the reaction mixture was allowed to react at 70° C. for additional 2.5 hours but the reaction was still incomplete. The composition of the reaction mixture was checked by HPLC (% by area): un-reacted noroxymorphone 9.7%, naltrexone 88.3%, 3-cyclopropylmethylnaltrexone 0.4%.

### Example 3

Noroxymorphone (40 g, KF 3.2%, assay 93.6%). (160 mL), potassium bicarbonate (18.3 g), water (1.06 mL) and cyclopropylmethyl bromide (15.2 mL) were charged in a 500 mL jacketed glass reactor. The mixture was heated up to 55° C. under vigorous stirring and maintained at 55° C. for 17 hours. The composition of the reaction mixture was checked by HPLC (% by area): noroxymorphone 0.8%, naltrexone 98.0%, 3-cyclopropylmethylnaltrexone 0.2%.

### Example 4

In a 500 mL reactor noroxymorphone (55.6 g. KF 7.48%, assay 90.0%), N-methyl-2-pyrrolidone (150 mL), potassium bicarbonate (24.4 g), water (2.7 g) and cyclopropylmethyl bromide (30.5 g) were mixed. The mixture was heated up to 55° C. and maintained under vigorous stirring for 16 hours. Excess of cyclopropylmethyl bromide was removed by distillation under vacuum. The reaction mixture was cooled down around 30° C. and diluted with water (250 mL) and HCl 37% (22.3 g). The composition of the reaction mixture was checked by HPLC (% by area), noroxymorphone 0.5%, naltrexone 97.5%, 3-cyclopropylmethylnaltrexone 0.5%. The obtained solution was further diluted with 1450 mL of water. The product was precipitated at 20-30° C. by adding drop-wise a solution of ammonium hydroxide 10% (66 g) up to pH 9.4. The product was filtered, washed with water (200 mL) and dried under vacuum overnight at 50° C. affording 59.4 g of naltrexone having an yield of 91.7% w/w. HPLC analysis (% by area): naltrexone 98.7%, noroxymorphone 0.1%, 3-cyclopropylmethylnaltrexone 0.5%.

### Example 5

Noroxymorphone (5.0 g, KF 3.34%) and potassium bicarbonate (2.0 g) were suspended in a mixture of N-methyl-2-pyrrolidone (19 mL) and water (1.0 mL). The mixture was heated up to 70° C. and cyclopropylmethyl bromide (2.73 g) was added in four portions over 3 hours. The reaction mixture was kept at 70° C. in total for 10.5 hours. The composition of the reaction mixture was checked by HPLC (% by area): noroxymorphone 4.6%, naltrexone 93.6%, 3-cyclopropylmethylnaltrexone 0.4%

### Example 6

A mixture of noroxymorphone (5.0 g, KF 3.34%), N-methyl-2-pyrrolidone (9.5 mL), water (9.5 mL) and potassium bicarbonate (2.0 g) was heated up to 70° C. Cyclopropylmethylbromide (2.73 g) is added in four portions over 1.5 hours. The mixture was maintained at 70° C. for 8 hours. The composition of the reaction mixture was checked by HPLC (% by area): unreacted noroxymorphone 93.7%, naltrexone 3.2%, 3-cyclopropylmethylnaltrexone 0.3%

### Example 7

A mixture of noroxymorphone (21 kg, KF 6.8%, assay 88%) and N-methyl-2-pyrrolidone (59 kg), potassium bicarbonate (9 kg), water (1.12 kg) and cyclopropylmethyl bromide (11.3 kg) was heated up to 55-58° C. Further cyclopropylmethylbromide (0.9 kg*2) was added to the mixture. After 29 hours (residual noroxymorphone 0.9% by HPLC area the reaction mixture was concentrated under vacuum at a maximum internal temperature of 80° C. collecting about 5 Kg of distillate. The mixture was then cooled to 30° C. and treated with a solution prepared mixing HCl 37% (7 kg) and water (92 kg). The solution was further diluted with water (536 Kg). The product was precipitated by addition of ammonium hydroxide 10% up to pH comprised in the range 9-10. Naltrexone was isolated by centrifugation, washed with water and dried under vacuum at 60° C. yielding 21.68 kg of product (KF 5%). HPLC analysis (% by area): naltrexone 98.5%, 3-cyclopropylmethylnaltrexone 0.45%)

TABLE 2
Schematic overview of water content and composition of
reaction mixture after alkylation.

| | | Wa- | Total | Composition of reaction | |
|---|---|---|---|---|---|
| | Water in | ter | amount | mixture after alkylation | |
| Exp. | noroxymorphone | added | of water | noroxymorphone | naltrexone |
| No. | (eq) | (eq) | (eq) | (A %) | (A %) |
| 1 | 0.4 | 0 | 0.4 | 4.5 | 94.0 |
| 2 | 0.55 | 0 | 0.55 | 9.7 | 88.3 |
| 3 | 0.55 | 0.45 | 1.0 | 0.8 | 98.0 |
| 4 | 1.33 | 0.86 | 2.2 | 0.5 | 97.5 |
| 5 | 0.55 | 3.3 | 3.9 | 4.6 | 93.6 |
| 6 | 0.55 | 31.4 | 31.9 | 93.7 | 3.2 |
| 7 | 1.23 | 0.97 | 2.2 | 0.9 | 97.6 |

1. A process for the manufacturing of naltrexone, comprising reacting noroxymorphone with cyclopropylmethyl halide in the presence of N-methyl-2-pyrrolidone and water, wherein the total amount of water compared to noroxymorphone is from 0.4 to 4.0 equivalents.

2. The process according to claim 1, wherein the total amount of water compared to noroxymorphone is from 0.7 to 3.5 equivalents.

3. The process according to claim 1, wherein the reaction takes place in the presence of an acid scavenger.

4. The process according to claim 3, wherein the acid scavenger is an inorganic or organic base or a mixture thereof.

5. The process according to claim 1, wherein the cyclopropylmethyl halide is cyclopropylmethyl bromide.

6. The process according to claim 1, wherein N-methyl-2-pyrrolidone is used in a weight by weight ratio of 0.5:1 to 10:1 with respect to noroxymorphone.

7. The process according to claim 3, wherein the molar relationship between noroxymorphone and the acid scavenger is from 1:0.5 to 1:2.

8. The process according to claim 1, wherein the molar relationship between noroxymorphone and cyclopropylmethyl halide is from 1:1 to 1:2.

9. The process according to claim 1, wherein the reaction temperature is in the range of 30-100° C.

10. The process according to claim 9, wherein the reaction temperature is in the range of 55-60° C.

11. The process according to claim 1, wherein the reaction is running for at least 8 hours.

12. The process according to claim 1, wherein the formed naltrexone is isolated by a method comprising the following steps
(a) concentrating the reaction mixture under vacuum;
(b) mixing the reaction mixture of step (a) with an acid;
(c) mixing the resulting mixture of step (b) with water;
(d) mixing the resulting mixture of step (c) with a base;
(e) isolating the resulting solid of step (d); and
(f) drying the solid.

13. The process according to claim 1, wherein the formation of 3-cyclopropylmethylnaltrexone is less than about 0.5% (by area).

14. The process according to claim 1, wherein the naltrexone obtained from the process is further processed to give nalmefene.

15. A process for the manufacturing of nalmefene comprising the following steps
(i) manufacturing naltrexone by the process according to claim 1; and
(ii) further processing the naltrexone obtained from step (i) to yield nalmefene.

16. The process according to claim 15, wherein the further processing of step (ii) comprises the Wittig reaction.

17. The process according to claim 16, further comprising precipitating nalmefene as a pharmaceutically acceptable salt.

18. The process according to claim 17, further comprising purifying the pharmaceutically acceptable salt of nalmefene.

19. The process according to claim 11, wherein the reaction is running in the range of 8-48 hours.

20. The process according to claim 19, wherein the reaction is running in the range of 16-20 hours.

21. The process according to claim 1, wherein the total amount of water compared to noroxymorphone is from 1.0 to 3.0 equivalents.
